(19) United States
(12) Patent Application Publication
(10) Pub. No.: US 2021/0039066 A1
(43) Pub. Date: Feb. 11, 2021

(54) Process of making substrate with activated carbon

(71) Applicant: Chin-San Hsieh, Tainan (TW)

(72) Inventor: Chin-San Hsieh, Tainan (TW)

(21) Appl. No.: 17/083,333

(22) Filed: Oct. 29, 2020

(51) Int. Cl. B01J 20/20; B01J 20/26; B01J 20/28; B01J 20/32; A61L 9/014; B01D 46/00; B01D 46/10

(52) U.S. Cl. CPC B01J 20/20; B01J 20/261; B01J 20/28028; B01J 20/3223; B01J 20/321; B01J 20/324; A61L 2209/22; A61L 9/014; B01D 46/0001; B01D 46/0038; B01D 46/0036; B01D 46/10; B01J 20/3297

(57) ABSTRACT

A process of producing a substrate having activated carbon includes solving an insoluble adhesive in water and mixing same to form an adhesive solution; adding activated carbon to the adhesive solution and mixing same until activated carbon is adhered to the adhesive solution to an activated carbon solution having activated carbon powder; adding the activated carbon solution to a substrate and flowing the activated carbon solution into the substrate; heating the substrate at a predetermined temperature for a predetermined time until water in the activated carbon solution of the substrate evaporate wherein vapor forms air channels toward outer surfaces of the substrate and the air channels are served as air passable holes of the substrate; and suddenly cooling the substrate having the air channels which are formed as the air passable holes, thereby producing a substrate having activated carbon.

### BACKGROUND OF THE INVENTION

### 1. Field of the Invention

The invention relates to processes of manufacturing substrate and more particularly to a process of producing a substrate having activated carbon, having air passable holes, capable of repetitively washing without activated carbon being lost, stable, capable of absorbing smell, water and sweat, and filtering out toxic particles.

### 2. Description of Related Art

Activated carbon is a form of carbon processed to have small, low-volume pores that increase the surface area available for adsorption or chemical reactions.

Activated carbon is used in air purification, solvent recovery, decaffeination, medicine, air filters in respirators, filters in compressed air, teeth whitening, production of hydrogen chloride and many other applications.

A conventional process of manufacturing substrate having activated carbon comprises the steps of mixing PVA, starch and water, heating same, cooling same to form a gel, adding catalyst (e.g., hydrochloric acid or formalin) and activated carbon to the gel, mixing same to form a solution, pouring the solution to a fibrous substrate, sending the substrate to an oven, heating the oven to foam the substrate, cooling the foamed substrate to a room temperature, washing the foamed substrate to a neutral substrate, pressing the neutral substrate to remove water and catalyst to form a dry substrate, and heating the dry substrate to a filtered substrate capable of absorbing smell and filtering out toxic particles.

The finished product is filtered substrate. The soluble sticky material (PVC) is used as element for adhering activated carbon. However, the filtered substrate and conventional process has the following drawbacks:

Activated carbon has a strong a strong attractive force. After activated carbon has mixed with the soluble sticky material, activated carbon is completely covered by the soluble sticky material. However, activated carbon may lose its properties including absorbing smell and filtering out toxic particles because there are no pores in the soluble sticky material. Thus, only an adhesion step is used to producing a filtered substrate. However, activated carbon is lost in the adhesion step. Thus, the product does not have any applications in daily life. Further, the lost activated carbon, having a dark color, may be dirty and pollute the environment.

The catalyst (e.g., chemical agent or hydrochloric acid) used in the producing process may pollute the environment.

A chemical reaction is undergone in the producing process in which the soluble sticky material is mixed with and starch. However, the chemical reaction is very slow and time consuming. Further, the soluble sticky material may be too sticky to produce the desired product since excessive time is taken in the mixing step.

Thus, it is desirable to provide an environmentally friendly process of manufacturing substrate having activated carbon capable of being permeable, repetitively washed, absorbing smell, filtering out toxic particles, and absorbing sweat.

### SUMMARY OF THE INVENTION

It is therefore one object of the invention to provide a process of manufacturing substrate having activated carbon capable of being permeable, repetitively washed, absorbing smell, filtering out toxic particles, and absorbing sweat.

To achieve above and other objects of the invention, the invention provides a process of producing a substrate having activated carbon comprising the steps of material preparation comprising solving an insoluble adhesive in water and mixing same to form an adhesive solution; activated carbon addition comprising adding activated carbon to the adhesive solution and mixing same until activated carbon is adhered to the adhesive solution to an activated carbon solution having activated carbon powder; substrate addition comprising adding the activated carbon solution to a substrate and flowing the activated carbon solution into the substrate; heating comprising heating the substrate at a predetermined temperature for a predetermined time until water in the activated carbon solution of the substrate evaporate wherein vapor forms a plurality of air channels toward outer surfaces of the substrate and the air channels are served as air passable holes of the substrate; and sudden cooling comprising suddenly cooling the substrate having the air channels which are formed as the air passable holes, thereby producing a substrate having activated carbon, having the air passable holes, capable of repetitively washing without activated carbon being lost, stable, capable of absorbing smell, and filtering out toxic particles.

Preferably the substrate is selected from the group consisting of fabric, resin cotton, polyethylene cotton, and non-fabric.

Preferably, the insoluble adhesive is formaldehyde free.

The process of manufacturing substrate having activated carbon and the substrate having activated carbon of the invention have the following advantageous effects in comparison with the prior art:

A solution having insoluble adhesive is used to join the activated carbon and the substrate. The insoluble adhesive cannot be solved in water in room temperature. When washing the substrate having activated carbon, activated carbon will not lose because activated carbon is adhered to the substrate. The vapor forms a plurality of air channels toward outer surfaces of the substrate and the air channels are served as air passable holes. Bores of the air passable holes are less than diameters of activated carbon. Thus, activated carbon is prevented from leaving the air passable holes by washing the substrate.

The insoluble adhesive is formaldehyde free for environment protection purpose. The process is physical in nature not a chemical reaction. Thus, its reaction time is short. No catalyst (e.g., hydrochloric acid or formalin) is required. The adhesive solution is prevented from been excessively sticky. Mass production is made possible. No chemical solvents are produced to pollute the environment. No additional step of removing acid for cleaning chemical solvents in the chemical reaction is required.

The produced substrate having activated carbon is capable of absorbing smell and filtering out toxic particles, being highly adhesive, being highly absorbing water, being highly absorbing sweat, being highly permeable due to the air channels and air passable holes, and being capable of repetitively washing without losing activated carbon.

The above and other objects, features and advantages of the invention will become apparent from the following detailed description taken with the accompanying drawings.

### BRIEF DESCRIPTION OF THE DRAWINGS

FIG. 1 is a flow chart of a process of manufacturing substrate having activated carbon according to the invention;

FIG. 2 is a perspective view of the substrate produced by the invention;

FIG. 3 is a sectional view taken along line A-A′ of FIG. 2; and

FIG. 4 is a detailed view of the area in a circle of FIG. 3.

### DETAILED DESCRIPTION OF THE INVENTION

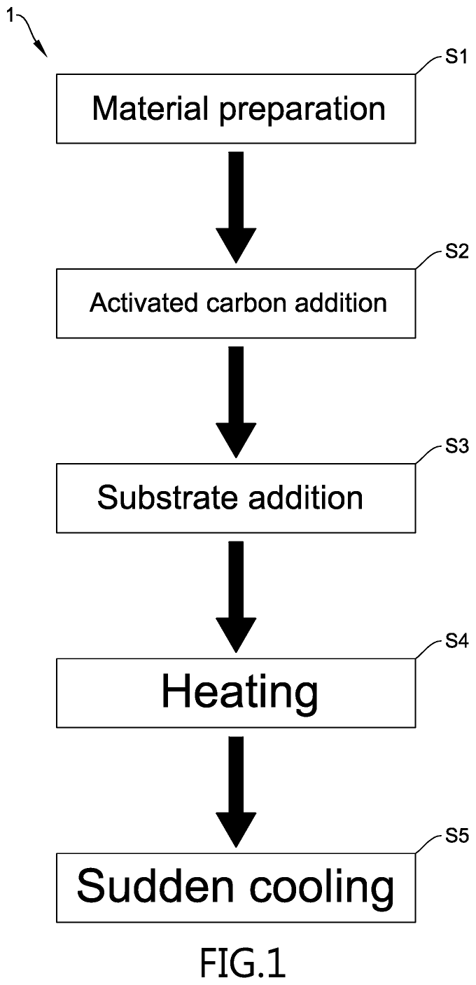

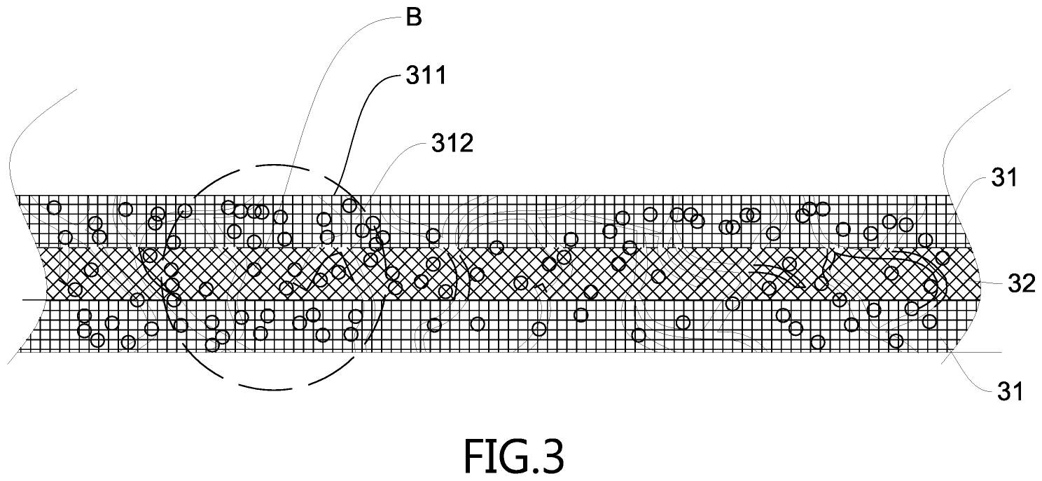

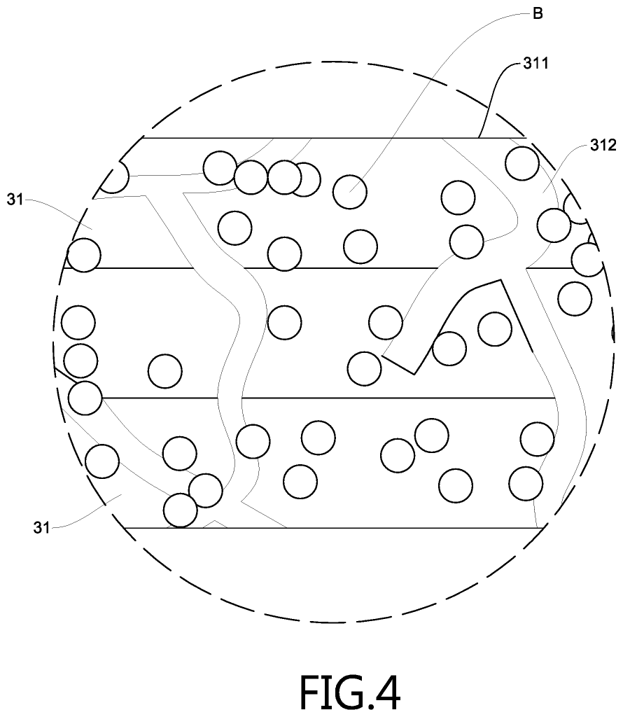

Referring to FIGS. 1 to 4, a process of manufacturing substrate having activated carbon in accordance with the invention is illustrated below. The process comprising the following steps:

S1 is a material preparation step and comprises solving an insoluble adhesive. It is noted that the insoluble adhesive will be solved in water before ripening and will be insoluble after ripening. Thus, the insoluble adhesive cannot be solved in water in room temperature. This step is used to prepare material before the insoluble adhesive is ripening. After ripening, sufficient water is added to the insoluble adhesive to form an adhesive solution. The insoluble adhesive is used to join the activated carbon and the substrate and adhesive is an insoluble adhesive in the invention. Prior to mixing the insoluble adhesive with activated carbon, the insoluble adhesive is solved in water to form a gel adhesive solution. Preferably, there is no formaldehyde in the insoluble adhesive for environmental protection purpose and preserving nature. That is, the insoluble adhesive is an insoluble adhesive fully complying with the environmental protection requirements.

S2 is activated carbon addition step and comprises adding activated carbon to the adhesive solution and mixing same. Activated carbon is adhered to the adhesive solution to an activated carbon solution having activated carbon powder. Activated carbon is capable of attracting surrounding material. Thus, activated carbon is adhered to the adhesive solution and surrounded by the adhesive solution after being mixed.

S3 is a substrate addition step and comprises adding the mixed activated carbon solution to a substrate. The activated carbon solution flows into the substrate in which an appropriate amount of activated carbon solution penetrates a predetermined thickness of the substrate based on needs. The substrate can be fabric, resin cotton, polyethylene cotton, or non-fabric. Preferably, the substrate is polyethylene. The substrate becomes wet and impermeable because the activated carbon solution penetration.

S4 is a heating step and comprises heating the substrate at 90-130° C. for a predetermined time until water in the activated carbon solution of the substrate evaporate in which the vapor may form a plurality of air channels 312 toward outer surfaces of the substrate and the air channels 312 are served as air passable holes 311.

S5 is a sudden cooling step and comprises suddenly cooling the substrate having the air channels 312 which are thus formed as the air passable holes 311. Bores of the air passable holes 311 are less than diameters of activated carbon because sizes of activated carbon are greater than water molecules. Thus, activated carbon is prevented from leaving the air passable holes 311 by washing the substrate. As a result, a substrate 3 having activated carbon, having the air passable holes 311, capable of repetitively washing without activated carbon being lost, stable, capable of absorbing smell, and filtering out toxic particles is produced.

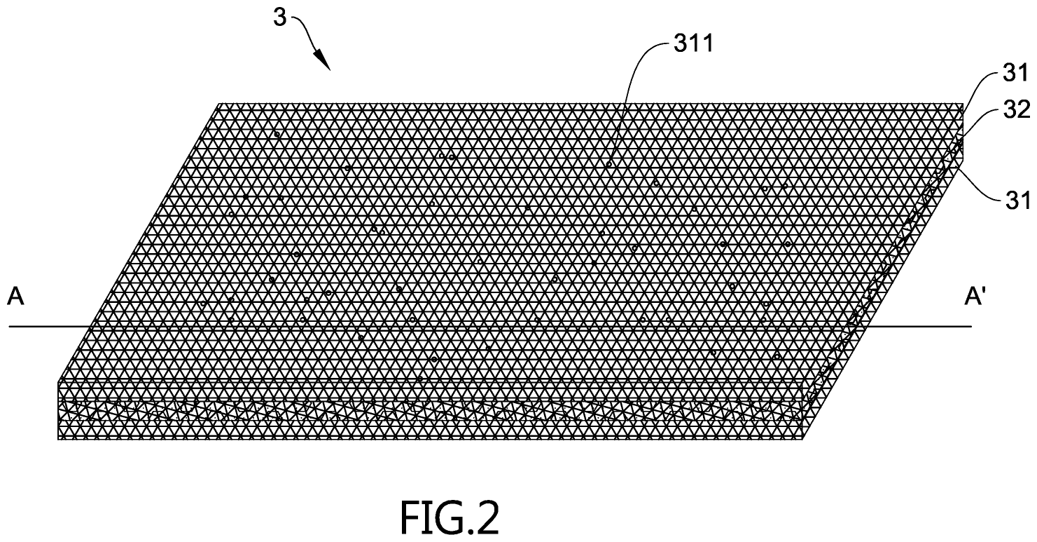

As shown in FIGS. 2, 3 and 4 specifically, a substrate 3 having activated carbon, produced by the producing process in accordance with the invention, comprises upper and lower substrate layers 31 and a activated carbon filter layer 32 between the upper and lower substrate layers 31 in which activated carbon powder B in the activated carbon filter layer 32 is adhered to the upper and lower substrate layers 31 using the insoluble adhesive. As such, the substrate 3 having activated carbon is formed. The substrate 3 having activated carbon comprises the upper and lower substrate layers 31 having the air channels 312 and the activated carbon filter layer 32 between the upper and lower substrate layers 31. The air channels 312 extend to the upper and lower substrate layers 31 to form the air passable holes 311 at the outer surfaces. Air passes through the air channels 312 to enter the air passable holes 311. Thus, the substrate 3 allows air to pass through. The insoluble adhesive cause portions of the activated carbon powder B to permeate the upper and lower substrate layers 31 and the activated carbon filter layer 32 in which portions of the activated carbon powder B adhere to outer surfaces of the air channels 312. Activated carbon will not lose by washing because bores of both the air channels 312 and the air passable holes 311 are less than diameters of activated carbon powder B. Thus, the substrate 3 having activated carbon can be washed repetitively without activated carbon being lost. As a result, a substrate 3 having activated carbon capable of absorbing smell and filtering out toxic particles, being highly adhesive, being highly absorbing water, being highly absorbing sweat, being highly permeable, and being capable of repetitively washing without losing activated carbon is produced.

Thickness, density and amount of activated carbon powder B of the upper and lower substrate layers 31 can be adjusted to an optimum based on requirements because activated carbon powder B is completely mixed in the upper and lower substrate layers 31. The substrate 3 having activated carbon can be applied to bed sheets, patient bed pads, shoe soles, cleaning pads, blinds, personal protective equipment, pads on dash boards, water filters, air filters, etc.

The process of manufacturing substrate having activated carbon and the substrate having activated carbon of the invention have the following advantageous effects in comparison with the prior art:

A solution having insoluble adhesive is used to join the activated carbon and the substrate. The insoluble adhesive cannot be solved in water in room temperature. When washing the substrate having activated carbon, activated carbon will not lose because activated carbon is adhered to the substrate. The vapor forms a plurality of air channels toward outer surfaces of the substrate and the air channels are served as air passable holes. Bores of the air passable holes are less than diameters of activated carbon. Thus, activated carbon is prevented from leaving the air passable holes by washing the substrate.

The insoluble adhesive is formaldehyde free for environment protection purpose. The process is physical in nature not a chemical reaction. Thus, its reaction time is short. No catalyst (e.g., hydrochloric acid or formalin) is required. The adhesive solution is prevented from been excessively sticky. Mass production is made possible. No chemical solvents are produced to pollute the environment. No additional step of removing acid for cleaning chemical solvents in the chemical reaction is required.

The produced substrate having activated carbon is capable of absorbing smell and filtering out toxic particles, being highly adhesive, being highly absorbing water, being highly absorbing sweat, being highly permeable due to the air channels and air passable holes, and being capable of repetitively washing without losing activated carbon.

While the invention has been described in terms of preferred embodiments, those skilled in the art will recognize that the invention can be practiced with modifications within the spirit and scope of the appended claims.

What is claimed is:

1. A process of producing a substrate having activated carbon comprising the steps of:
material preparation comprising solving an insoluble adhesive in water and mixing same to form an adhesive solution;
activated carbon addition comprising adding activated carbon to the adhesive solution and mixing same until activated carbon is adhered to the adhesive solution to an activated carbon solution having activated carbon powder;
substrate addition comprising adding the activated carbon solution to a substrate and flowing the activated carbon solution into the substrate;
heating comprising heating the substrate at a predetermined temperature for a predetermined time until water in the activated carbon solution of the substrate evaporate wherein vapor forms a plurality of air channels toward outer surfaces of the substrate and the air channels are served as air passable holes of the substrate; and
sudden cooling comprising suddenly cooling the substrate having the air channels which are formed as the air passable holes, thereby producing a substrate having activated carbon, having the air passable holes, capable of repetitively washing without activated carbon being lost, stable, capable of absorbing smell, and filtering out toxic particles.

2. The process of claim 1, wherein the substrate is selected from the group consisting of fabric, resin cotton, polyethylene cotton, and non-fabric.

3. The process of claim 1, wherein the insoluble adhesive is formaldehyde free.
